(19) United States
(12) Patent Application Publication
(10) Pub. No.: US 2011/0318732 A1
(43) Pub. Date: Dec. 29, 2011

(54) PREDICTION OF CHEMOTHERAPEUTIC RESPONSE VIA SINGLE-CELL PROFILING OF TRANSCRIPTION SITE ACTIVATION

(76) Inventors: Robert H. Singer, New York, NY (US); Rosanna Pezo, Bronx, NY (US); Leonard H. Augenlicht, Redding, CT (US)

(21) Appl. No.: 12/736,885

(22) PCT Filed: May 7, 2009
(86) PCT No.: PCT/US2009/002819

§ 371 Date: May 11, 2011

Related U.S. Application Data
(60) Provisional application No. 61/130,079, filed on May 28, 2008.

(51) Int. Cl. C12Q 1/68

(52) U.S. Cl. 435/6.11

(57) ABSTRACT

The present invention generally relates to methods for determining tumor resistance or sensitivity to chemotherapeutic agents and the likelihood of tumor reoccurrence based on the expression levels of genes known to correlate to the chemotherapeutic agent. In particular, the expression levels of TYMS, MRGX, ATP7B and/or BAK in tumor cells, as measured by the number of active transcription sites detected by fluorescence in situ hybridization (FISH), are predictive of resistance and sensitivity to chemotherapy and the likelihood of reoccurrence following chemotherapy treatment.

### CROSS-REFERENCE TO RELATED APPLICATION

This application claims the benefit of U.S. Provisional Patent Application No. 61/130,079, filed May 28, 2008, the content of which is hereby incorporated by reference into the subject application.

### STATEMENT OF GOVERNMENT SUPPORT

The invention disclosed herein was made with U.S. Government support under Grant Number CA83208 from The National Institutes of Health and MSTP Training Grant Number T32GM07288. Accordingly, the U.S. Government has certain rights in this invention.

### FIELD OF THE INVENTION

The present invention generally relates to methods for determining tumor resistance or sensitivity to chemotherapeutic agents and the likelihood of tumor reoccurrence based on the expression levels of genes known to correlate to the chemotherapeutic agent. In particular, the expression levels of TYMS, MRGX, ATP7B and/or BAK in tumor cells, as measured by the number of active transcription sites detected by fluorescence in situ hybridization (FISH), are predictive of resistance and sensitivity to chemotherapy and the likelihood of reoccurrence following chemotherapy treatment.

### BACKGROUND OF THE INVENTION

Throughout this application various publications are referred to by Arabic numerals in parentheses. Full citations for these references may be found at the end of the specification immediately preceding the claims. The disclosures of these publications are hereby incorporated by reference in their entireties into the subject application to more fully describe the art to which the subject application pertains.

5-Fluorouracil (5-FU) is the most commonly used agent in combination therapy for colorectal cancer in either an adjuvant or advanced stage setting (1). While stage is a significant predictor of likely outcome, cellular and molecular markers of sensitivity to 5-FU, or disease free or overall survival, have been identified for each stage. Among these are levels of thymidylate synthase and thymidine phosphorylase, two enzymes intimately related to 5-FU metabolism (2-4). The presence of microsatellite instability has also been linked to 5-FU response (5, 6). Finally, the presence of a wild-type p53 gene (7-9), especially when coupled with amplification and/or elevated expression of the c-myc gene (10, 11), correlates with a favorable response to 5-FU.

More recently, unbiased approaches that utilize gene expression profiling have characterized response to drugs and prognosis. With regard to colorectal cancer, heterogeneous responses to 5-FU (12), camptothecin (12), and oxaliplatin (13) were identified in a panel of 30 cell lines, and microarray analysis was used to identify gene expression profiles predictive of relative sensitivity to these drugs.

Regardless of the method used to identify clinically useful markers of drug response, all approaches must eventually deal with the fact that tumors are highly heterogeneous. Only a minor proportion of the cells may be relatively drug resistant or have other important clinical phenotypes, such as propensity to invade or metastasize. Since these cells cannot be identified histologically, alternate means are necessary for their detection. This is not only of major clinical importance, but the distribution of such cells in relation to important features of the tumor, such as the invasive front or the proximity to blood supply, provide significant insight into the cell biology of tumor formation and progression. While immunohistochemistry can provide such information, it is limited by the availability of appropriate antibodies, as well as in the number of distinct gene products that can be identified simultaneously.

Accordingly, there is a pressing need for an improved method for predicting tumor response to chemotherapy and the likelihood of tumor reoccurrence. The present invention satisfies this need.

### SUMMARY OF THE INVENTION

The present invention is directed to methods for predicting resistance or sensitivity of a tumor to a chemotherapeutic agent by determining the level of expression in a tumor cell for genes that correlate to the chemotherapeutic agent (e.g. TYMS and MRGX for tumor resistance and ATP7B and BAK for tumor sensitivity). The present invention is also directed to a method for determining the likelihood of tumor reoccurrence.

### BRIEF DESCRIPTION OF THE FIGURES

FIGS. 1A-1C. Defining markers of 5-FU response in human colorectal tumor cell lines using single-cell profiling of transcription site activation. A) Flowchart of the strategy used to define a predictive model for response to 5-FU-based chemotherapy. Candidate genes were selected from gene expression profiles of each human colorectal adenocarcinoma cell line. The training set of cell lines selected represents the extremes of sensitivity or resistance to 5-FU. A transcription site activation profile of candidate genes was determined for each cell line. Using leave-one-out analysis, a predictive model that classified the training set of cell lines as resistant or sensitive to 5-FU with the highest accuracy was derived. The predictive marker genes were evaluated for their ability to accurately classify a panel of independent test cell lines as 5-FU resistant or sensitive in a blinded study. B) Detection of an active transcription site for the gene MRGX in an individual human colorectal adenocarcinoma cell (DLD-1). Nuclei are stained with DAPI and sites of transcription are detected with fluorescent probes labeled in Cy3 and Cy5 (colors not shown). Inset shows close-up of area of nucleus with both Cy3 and Cy5 probes bound to nascent transcripts. Scale bar, 6 microns. C) Transcription site activation profile of 5-FU resistant and 5-FU sensitive colorectal tumor cell lines as measured by FISH for nascent mRNAs. Analysis of active transcription sites for each candidate gene in individual cells provides a transcriptional profile for each cell line. Candidate genes correlated with 5-FU resistance are represented by light shading; candidate genes correlated with 5-FU sensitivity are represented by dark shading. Data represent the mean±s.e.m. for three experiments.

FIGS. 2A-2C. Chemotherapy indicator plot. A) Two genes that are poor predictive markers of response to 5-FU treatment. Cell lines known to be sensitive represented by filled squares. Cell lines known to be resistant represented by filled circles. The decision line is an average of 12 decision boundaries generated from leaving out each of the 12 samples from the training set once. The large error in the decision boundary signifies the dependency of the model on a single sample in the training set. B) The four genes, MRGX, TYMS, BAK and ATP7B are identified as good predictive markers of response to 5-FU treatment. C) Performance of biomarkers in an independent set of blinded test cell lines. Test cell lines A and D, corresponding to RKO and HCT116, respectively, were classified as 5-FU-sensitive (P=0.05 and P=0.0005, respectively). Test cell line B, corresponding to SW620 was classified as 5-FU-resistant (P=0.023). The fourth cell line, HCT15 was also classified as 5-FU resistant (P=0.099).

FIGS. 3A-3D. Detection of active transcription sites for 5-FU marker genes in paraffin-embedded human colon tumor TMA. A) Merge of DAPI, Cy3 and Cy5 channels. Image shows DAPI-stained nuclei containing transcription sites (arrows) for MRGX and TYMS. Scale bar, 5 microns. B) Merge of DAPI, Cy3 and Cy5 channels. Image shows DAPI-stained nuclei containing transcription sites (arrows) for ATP7B and BAK. Scale bar, 5 microns. C) Active transcription site profile for 5-FU marker genes in colon tumor biopsies from individual patients as measured by RNA FISH. Genes correlated with 5-FU resistance are TYMS and MRGX. Genes correlated with 5-FU sensitivity are ATP7B and BAK. Data represent the mean±s.e.m. for three sections from each individual tumor. D) Chemotherapy indicator plot for tumors from 15 anonymous patients. Tumor samples with unknown clinical outcomes represented by filled diamonds. The predictive model classified 11 samples as sensitive and 2 samples as resistant. The model was unable to classify the remaining two samples with significant confidence.

FIGS. 4A-4C. Prediction of response to 5-U-based chemotherapy in colon cancer patients. A) Active transcription sites for 5-FU marker genes in paraffin-embedded human colon tumor tissues. Image shows DAPI-stained nuclei containing transcription sites for ATP7B and BAK (color not shown). Scale bar, 5 microns. B) Active transcription site profile for 5-FU marker genes in colon tumor samples from seven patients as measured by RNA FISH. The seven patients are designated as follow: Patient #1F, female age 60, tumor stage T3N2Mx (“Poorly differentiated adenocarcinoma”); Patient #4F, male age 56, tumor stage T3N1Mx (“Poorly differentiated mucinous adenocarcinoma”); Patient #6F, male age 33, unknown tumor stage (“Metastastic adenocarcinoma”); Patient #1N, male age 62, tumor stage T3N1Mx (“Well to moderately differentiated adenocarcinoma”); Patient #4N, female age 67, tumor stage T3N2Mx (“Moderately differentiated adenocarcinoma”); Patient #5N, female age 56, tumor stage T3N2Mx (“Moderately to poorly differentiated adenocarcinoma”); Patient #6N, female age 42, tumor stage T3N1Mx (“Moderately differentiated adenocarcinoma”). Genes correlated with 5-FU resistance are represented by light shaded bars. Genes correlated with 5-FU sensitivity are represented by dark shaded bars. Data represent the mean±s.e.m. for six fields from each individual tumor. C) Chemotherapy indicator plot for tumors from seven anonymous patients. Patients known to be sensitive represented by filled squares. Patients known to be resistant represented by filled circles. The predictive model classified 3 samples as resistant and 4 samples as sensitive.

### DETAILED DESCRIPTION OF THE INVENTION

The present invention provides a method for predicting resistance of a tumor to a chemotherapeutic agent comprising determining the level of expression of TYMS and/or MRGX, wherein a high level of expression of TYMS and/or MRGX indicates that the tumor is resistant to the chemotherapeutic agent. As used herein, “tumor resistance” refers to the ability of the cells of the tumor to survive treatment with a chemotherapeutic agent. A tumor with high resistance comprises a large number of cells that are able to survive chemotherapeutic treatment.

The present invention further provides a method for predicting sensitivity of a tumor to a chemotherapeutic agent comprising determining the level of expression of ATP7B and/or BAK, wherein a high level of ATP7B and/or BAK indicates that the tumor is sensitive to the chemotherapeutic agent. As used herein, “tumor sensitivity” refers to the ability of the cells of the tumor to respond favorably to chemotherapeutic agent (i.e. the ability of the chemotherapeutic cells to induce apoptosis in a majority of the tumor cells). A tumor with high sensitivity comprises a large number of cells that do not survive chemotherapeutic treatment.

The methods provided by the present invention can be applied to any tumor. Tumors of the human body (e.g. prostate, lung, colorectal, skin, pancreas, breast, ovarian, etc.) are well known in the art. In the preferred embodiment, the tumor is a human colorectal tumor.

In accordance with the present invention, the level of expression is determined by the number of active transcription sites for TYMS and/or MRGX for predicting tumor resistance and ATP7B and/or BAK for predicting tumor sensitivity. In the preferred embodiment, the number of active transcription sites is determined via fluorescence in situ hybridization. Preferably, the active transcription sites are located in the interphase nucleus of the cell.

Chemotherapeutic agents are well known in the art for treating tumors. These include alkylating agents, antimetabolites, anthracyclines, plant alkaloids, topoisomerase inhibitors, and other antitumor agents. In the preferred embodiment, the chemotherapeutic agent is 5-Fluorourcil (5-FU).

The present invention further provides a method for predicting resistance of a tumor to a chemotherapeutic agent comprising determining the level of expression in a cell of the tumor for TYMS and/or MRGX, and for ATP7B and/or BAK, wherein a higher level of expression for TYMS and/or MRGX compared to the level of expression for ATP7B and/or BAK indicates that the tumor is resistant to the chemotherapeutic agent.

Additionally, the present invention provides a method for predicting sensitivity of a tumor to a chemotherapeutic agent comprising determining the level of expression in a cell of the tumor for TYMS and/or MRGX, and for ATP7B and/or BAK, wherein a lower level of expression for TYMS and/or MRGX compared to the level of expression for ATP7B and/or BAK indicates that the tumor is sensitive to the chemotherapeutic agent.

In one embodiment, the method for predicting the resistance or sensitivity of a tumor to a chemotherapeutic agent comprise determining the level of expression for TYMS, MRGX, ATP7B and BAK. Resistance or sensitivity to the chemotherapeutic agent can then be predicted based on the levels of expression. For example, a cell having higher levels of expression for TYMS and MRGX compared to the levels of expression for ATP7B and BAK indicate that the tumor is likely to be resistant to the chemotherapeutic agent. Conversely, higher levels of expression for ATP7B and BAK compared to the levels of expression for TYMS and MRGX indicate that the tumor is likely to be sensitive to the chemotherapeutic agent.

Preferably, the level of expression for TYMS and/or MRGX, and for ATP7B and/or BAK is determined by the number of active transcription sites in the tumor cell for TYMS and/or MRGX, and for ATP7B and/or BAK. In accordance with the present invention, the number of active transcription sites is determined via fluorescence in situ hybridization. Preferably, the active transcription sites are located in the interphase nucleus of the cell.

The present invention further provides a method for determining the likelihood of tumor reoccurrence following treatment with a chemotherapeutic agent. For example, one skilled in the art can determine the level of expression in a cell of the tumor for TYMS and/or MRGX, wherein a high level of expression for TYMS and/or MRGX indicates that the tumor is likely to reoccur following treatment with a chemotherapeutic agent. In another example, one skilled in the art can determine the level of expression in a cell of the tumor for ATP7B and/or BAK, wherein a high level of expression for ATP7B and/or BAK indicates that the tumor is likely not to reoccur following treatment with a chemotherapeutic agent.

The present invention also provides a method for determining the likelihood of tumor reoccurrence following treatment with a chemotherapeutic agent which comprises determining the level of expression in a cell of the tumor for TYMS and/or MRGX and for ATP7B and/or BAK, wherein a higher level of expression for TYMS and/or MRGX in the cell compared to the level of expression for ATP7B and/or BAK in the cell indicates that the tumor is likely to reoccur following treatment with the chemotherapeutic agent. Conversely, a higher level of expression for ATP7B and/or BAK compared to the level of expression for TYMS and/or MRGX indicates that the tumor is likely not to reoccur following treatment with a chemotherapeutic agent.

In one embodiment, the method for determining the likelihood of tumor reoccurrence following treatment with a chemotherapeutic agent comprises determining the level of expression for TYMS, MRGX, ATP7B and BAK. The likelihood of tumor reoccurrence following treatment with a chemotherapeutic agent can then be predicted based on the levels of expression. For example, a cell having higher levels of expression for TYMS and MRGX compared to the levels of expression for ATP7B and BAK indicate that the tumor is likely to reoccur. Conversely, higher levels of expression for ATP7B and BAK compared to the levels of expression for TYMS and MRGX indicate that the tumor is likely to not reoccur.

The present invention further provides a method for determining the resistance or sensitivity of a tumor to a chemotherapeutic agent comprising (a) determining the level of expression in a cell of the tumor for a gene or genes known to correlate in response to the chemotherapeutic agent and (b) comparing the level of expression determined in step (a) with the level of expression in a cell of a control tumor having a known resistance or sensitivity to the chemotherapeutic agent, wherein the resistance or sensitivity of the tumor to the chemotherapeutic agent is similar to that of the control tumor if the level of expression of the gene or genes in the cell of the tumor is similar to that of the cell of the control tumor. As used herein, a gene that “correlates” in response to a chemotherapeutic agent refers to a gene which exhibits a change in expression (i.e., an increase or decrease in expression) in response to treatment of the tumor with a chemotherapeutic agent. In accordance with the present invention, a control tumor may be obtained from an individual who has previously been treated with the same chemotherapeutic agent as that being used to treat the subject tumor, wherein the individual's response to the chemotherapeutic agent has been examined.

Methods for identifying a gene or genes that correlate in response to a chemotherapeutic agent are well known in the art (e.g., Northern blotting, real-time polymerase chain reaction (RT-PCR), expression profiling, etc.). In accordance with the present invention, the gene or genes may be identified via microarray.

This invention will be better understood from the Experimental Details, which follow. However, one skilled in the art will readily appreciate that the specific methods and results discussed are merely illustrative of the invention as described more fully in the claims that follow thereafter.

### Experimental Details

### Materials and Methods

Cell culture. DLD, HCT15, SW837, SW620, HCT116, RW2982, and SW403 cell lines with documented responses to 5-FU were provided by J M Mariadason (Montefiore Medical Center, Bronx, N.Y.), grown in MEM (Cellgro), supplemented with 10% FBS (Invitrogen), 1% penicillin/streptomycin (Invitrogen), 100 uM nonessential amino acids (Sigma), and 10 mM HEPES buffer (Invitrogen) in a humidified incubator at 37° C. with 5% CO₂.

Oligonucleotide probe design and synthesis. Probes for FISH were designed using OLIGO-6.0 software (Molecular Biology Insights) and specificity was verified through the NCl GeneBank nucleotide-nucleotide BLAST program. For each target nascent transcript, four 50-mer DNA probes were synthesized containing 4-5 modified thymidine bases conjugated to either Cy3 or Cy5 succinimidyl ester fluorescent dyes (GE Healthcare).

Patient tissue samples. Tissue microarrays (TMA) containing core biopsies of paraffin-embedded tissues from 15 anonymous colon cancer patients in triplicate were purchased (US Biomax). Paraffin-embedded tissue samples with known outcomes were obtained from seven patients who had undergone treatment for colon cancer at the Kimmel Cancer Center, Thomas Jefferson University, Philadelphia, Pa., as follow. RNA FISH in cultured cells and paraffin-embedded tissues. Cells were grown on glass coverslips, extracted with Triton X-100, fixed with 4% paraformaldehyde and hybridized with 20 ng of labeled probe as described (16). Paraffin-embedded tissue FISH was performed as described (17).

Detection of transcription sites. Fluorescent signals were detected with an epifluorescence Olympus AX70 microscope, UApo 40×, 1.35NA and PlanApo 60×, 1.4NA objectives, and a CoolSNAP-HQ CCD camera (Photometrics) using filters for DAPI (#SP100), FITC (#SP101), Cy3 (#SP-102v2), and Cy5 (#SP104v2) (Chroma Technology). Stacks of images were acquired with a 200 nm Z step size and analyzed using IPLab software version 3.61 (BD Biosciences). Random fields of cells were imaged to ensure that differences in numbers of active transcription sites between samples were due to differences in transcription and not due to heterogeneity in proliferation among cells within a culture or tissue sample. Transcription sites were assayed in untreated cell cultures and tissues except for samples from patients 1F, 4F and 6F, who received 5-FU therapy prior to surgical resection of their tumors. Only nuclei located entirely within the imaged field were scored for presence or absence of transcription sites. Each image within a stack was analyzed separately to accurately count nuclei in close proximity. Fluorescent spots in the nucleus were identified as transcription sites based on fluorescence intensity, volume, and shape. Spots also present in the FITC channel represented autofluorescence and were not counted. Transcripts were first detected individually, using Cy3 and Cy5 probes. After identifying a four gene signature predictive of 5-FU response, the genes were analyzed simultaneously in the same sample. Two genes correlating with resistance (TYMS and MRGX) were detected with probes labeled with one fluorophore and two genes correlating with sensitivity (ATP7B and BAK) were detected with probes labeled with a different fluorophore. Percentage of transcription sites for each gene was calculated from the total number of transcription sites detected and the total number of nuclei detected.

Statistical analysis. Statistical tests were performed using MATLAB v7.0.1 (MathWorks). To perform logistic regression, P was assigned the probability that a cell line is sensitive to 5-FU, given its gene expression profile X=[x₁, . . . , xₙ], where xₙ is the percentage of cells containing transcription sites for gene n in cell line x. The odds of sensitivity to 5-FU are P/(1−P). The odds were parameterized such that

$$\begin{matrix}ln ( \frac{P}{1 - P} ) = {w}_{0} + {X}^{T} {w}_{1} & eq . 1 \\ ∴ P = \frac{{e}^{{w}_{0} + {X}^{T} {w}_{1}}}{1 + {e}^{{w}_{0} + {X}^{T} {w}_{1}}} & eq . 2\end{matrix}$$

where X^{T} denotes the transpose of X. A maximum likelihood estimator was utilized that uses as an input the measured quantities xₙ and outcomes for each of the training samples. The estimator then iteratively solved for P by varying the parameters w₀ and w₁. The linear decision boundary could then be written as

$$ln ( \frac{P}{1 - P} ) = {w}_{0} + {X}^{T} {w}_{1} = 0 \qquad eq . 3$$

Primer Sequences.

| MRGX (MORF-related protein X) NM_012286 | |
|---|---|
| | (SEQ ID NO: 1) |
| 1 | TTTTCTGATGGTGACCTGAAACGAGAATCCAGATCTTCCCAGCAGCCGAC |
| | (SEQ ID NO: 2) |
| 2 | GCAGATTGCTGTCCACGAGGTTGAGAACCCTGCTTTCTGGAACTCATTCA |
| | (SEQ ID NO: 3) |
| 3 | CCTCCATTCTATTCTTAAACGCCTCACTTTCAACAGTGGGGTCTGCC |
| | (SEQ ID NO: 4) |
| 4 | AGGATTTCAGCATACTGGGGCCTCTCAAATTTGTAGAGCAGCTGAGTGCC |
| TYMS (thymidylate synthase) NM_001071 | |
| | (SEQ ID NO: 5) |
| 1 | CCTCCAAAACACCCTTCCAGAACACACGTTTGGTTGTCAGCAGAGGGAAT |
| | (SEQ ID NO: 6) |
| 2 | ATCTCTGTATTCTGCCCCAAAATGCCTCCACTGGAAGCCATAAACTGGGC |
| | SEQ ID NO: 7) |
| 3 | CGAAGAATCCTGAGCTTTGGGAAAGGTCTGGGTTCTCGCTGAAGCTGAAT |
| | (SEQ ID NO: 8) |
| 4 | GGCATCCAGCCCAACCCCTAAAGACTGACAATATCCTTCAAGCTCCTTTG |
| BAK (BCL2-antagonist/killer 1) NM_001188 | |
| | SEQ ID NO: 9) |
| 1 | CTGCTGATGGCGGTAAAAAACGTAGCTGCGGAAAACCTCCTCTGTGTCCT |
| | (SEQ ID NO: 10) |
| 2 | GGCACCCTTGGGAGTCATGATTTGAAGAATCTTCGTACCACAAACTGGCC |
| | (SEQ ID NO: 11) |
| 3 | CTTCTCCCACTTAGAACCCTCCAGATGAACTCCCTACTCCTTTTCCCTGA |
| | SEQ ID NO: 12) |
| 4 | AGGGGATTGCACAGTTTATTTCCAAACACTCAGAGGATAGGGGGTGGCCT |
| ATP7B (ATPase, Cu²⁺ transporting, β polypeptide) NM_000053 | |
| | (SEQ ID NO: 13) |
| 1 | GGCCAGGCCATCCAGACCACCTTCATAGCCAACATTGTCAAAAGCAAAAC |
| | (SEQ ID NO: 14) |
| 2 | TCCGCCTTCTCAGCCACAGCAACCACCAGGATGACCAGAGAATAAACATA |
| | (SEQ ID NO: 15) |
| 3 | GGAAGTCCGTGCAGTATCCCAAGGTCTCTGTTCCAAGTTCCTCTTTACAG |
| | (SEQ ID NO: 16) |
| 4 | GGTCCCCACTGACAAGCACACAGGAGAGAAAAGGAACAGACTATGTACGA |
| PPP4R1 (protein phosphatase 4, regulatory subunit 1) NM_005134 | |
| | (SEQ ID NO: 17) |
| 1 | ATCTCTTTCATCATCGCAGACTTCCCTCAAGGTATCGAGCAAACTCCGGG |
| | (SEQ ID NO: 18) |
| 2 | TCTGGCCTGACTTGTACATCCTCTGGGGCTTCTTGATCTCTGGTCCTATT |
| | (SEQ ID NO: 19) |
| 3 | GTAAAGAGCTGTCCAGTGGAACACTAATTTCACCTAGAGGCTTCCCGGAT |
| | (SEQ ID NO: 20) |
| 4 | GTGCTTAGCAATTTCAGTGTCAACCGTCTGTGCACGAGAAGGGTCAGTCA |
| FLJ22474 NM_024719 | |
| | (SEQ ID NO: 21) |
| 1 | TTAAACAAACAGTCCCAGATCCGAAGCACTGTCTCCACGGGCAAGATGTC |
| | (SEQ ID NO: 22) |
| 2 | GTGGCTTCCAAAATCAACTCCTGGTGCTGCTTAATTAAGGTCAGGGCCAC |
| | (SEQ ID NO: 23) |
| 3 | ACACTTCTGGCACACACAGCAGATGAGTACAGCCATTAGGACCAGGA |
| | (SEQ ID NO: 24) |
| 4 | TCTCTTTGATGAAGGCCCAGCTGCTGAAATACCGCCCACGTTTGCCATG |
| ATUB2 (tubilin, α2) NM_006001 | |
| | (SEQ ID NO: 25) |
| 1 | ACTTGGCATCTGACCATCGGGCTGAATTCCATGTTCCAGGCAGTACAGTT |
| | (SEQ ID NO: 26) |
| 2 | TGGAGACCTGGGGGGCTGGGTAAATGGCAAATTCTAGCTTGGACTTCTTG |
| | (SEQ ID NO: 27) |
| 3 | TCACACTTGACCATCTGATTGGCTGGCTCGAAGCAGGCATTGGTGATCTC |
| KIDDNS NM_020738 | |
| | (SEQ ID NO: 28) |
| 1 | CGGTGCTCCAAGTTAACCCCACATTTCAGTAGTTCCTCTACGATGTGCAC |
| | (SEQ ID NO: 29) |
| 2 | TATGTCTTGCCAATCTAGTGCTGAGGACCCAGGCCCAATTCCAACTCTCT |
| | (SEQ ID NO: 30) |
| 3 | CTCCCCAAGGCTGTTCTGTGAAACTCTTCTGTCAGCATCTTCCTGTATCC |
| | (SEQ ID NO: 31) |
| 4 | TATTGTTGTTCAGAGTCACGGTGCTGGGAGTTCGGTTCAGGTTGTAGGCT |
| PB1 (polybromol) NM_018165 | |
| | (SEQ ID NO: 32) |
| 1 | GCCCTCATCTCACTGCTGAACAGGATGTAGCCACTCATGTTGATTTTCCG |
| | (SEQ ID NO: 33) |
| 2 | TCTTTGGTGGGGCAGCTACAAACATGGGTGTTGTTGGCTGCTGTATGACA |
| | (SEQ ID NO: 34) |
| 3 | TTGTATGCTTGGCGAATGTTGAGGGTGTCCCGGAGCATCAAATCTCGAAG |
| | (SEQ ID NO: 35) |
| 4 | ACATGCCGCCAAGTGAAACAATGTTCCTACTGTCTGCCATCCTATGCTGC |
| PRSS (protease, serine 15) NM_004793 | |
| | (SEQ ID NO: 36) |
| 1 | ATAAGGCTGGGCGAGACGAACTTTCCTTCTCAGCAGCTCAACCAACTTCT |
| | (SEQ ID NO: 37) |
| 2 | TGTAGAGAGGGTTCAAGGCAATGATGTCCCGGATGGTCTTCACGATCTCT |
| | (SEQ ID NO: 38) |
| 3 | TCGATGAGGATCAGGGGGTTCTCCGTCTTGGTCTTCAAACACTGGAT |
| | (SEQ ID NO: 39) |
| 4 | TCTTGTAGGCCGATTTCCGTAACACCTTCTCCACTTGCTTCTGCAGGTTG |
| NUCB2 (nucleobindin 2) NM_005013 | |
| | (SEQ ID NO: 40) |
| 1 | AGAGAAAGCAATACTGTAGCAGGATGGTCCTCCACCTCATGTTCAGGCAG |
| | (SEQ ID NO: 41) |
| 2 | CCTCTGTGAAGAACTGTTGCTGATCTAATGTCTCCCAGCTATCTGGCTCC |
| | (SEQ ID NO: 42) |
| 3 | ATGACCTGATGATATTCCAGCTTCTGAGCCTCCAGTTGATCATGCTGACG |
| | (SEQ ID NO: 43) |
| 4 | CAGACTTTAAATGTGTGGCTCAAACTTCAATTCTCCAGCTGGCCCTGATG |
| TSSC3 (tumor suppressing, subtransferable candidate 3) NM_003311 | |
| | (SEQ ID NO: 44) |
| 1 | AAGTCGATCTCCTTGTGGTCGGTGGTGACGATGGTGAAGTACACGTACTT |
| | (SEQ ID NO: 45) |
| 2 | TATTAGATAGTCCAATAACTTAAGGCGCCCGTGCAACGGAGCGAGGATCC |
| | (SEQ ID NO: 46) |
| 3 | TCTCACTGAGCCACAGCCGGATGGTAGAAAAGCAAACTGGCCAAGTGATT |
| | (SEQ ID NO: 47) |
| 4 | ATTCATTTATTCATTCAAAGCCGGTTCCCAGCGCCTTTCACACCAGCCCC |

### Results

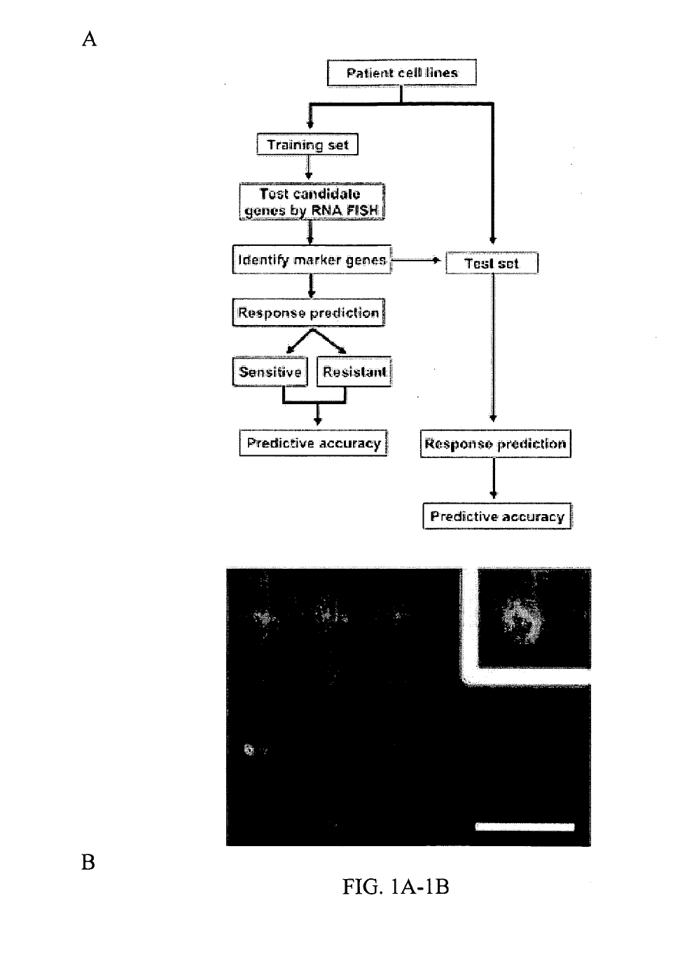

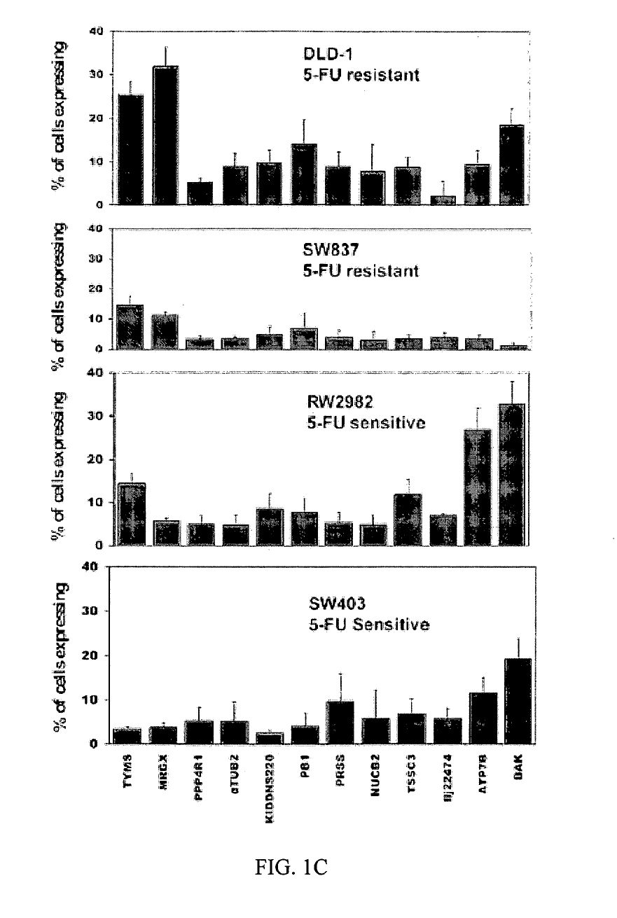

To develop markers predictive of 5-FU response, four colorectal adenocarcinoma cell lines representing extremes of sensitivity or resistance to 5-FU were selected and a set of candidate genes including thymidylate synthase and genes that correlated highly with 5-FU response in a microarray study were chosen (12). FIG. 1A provides an overview of the strategy. For each of the 12 candidate genes, active transcription sites in individual cells were examined using FISH (FIG. 1B). The results demonstrated differential transcription of several genes in 5-FU-sensitive or resistant colorectal tumor cell lines (FIG. 1C). Various combinations of these genes were examined to identify expression signatures that correlated with either resistance or sensitivity to 5-FU.

To evaluate the predictive value of each combination of genes, logistic regression was used to build a model that predicted response of a cell line to 5-FU based on the active transcription site profile of those genes. Exhaustive combinations of the twelve potential markers for 5-FU response were used to build various models, each of which was evaluated for predictive accuracy using a training set of four cell lines with documented responses to 5-FU (12).

Due to the small sample size of the training set, leave-one-out cross-validation was used to assess the accuracy of the predictive models. The transcriptional profile and the outcome of k−1 of the k training samples was used to produce a linear decision boundary as outlined in the statistical methods section. The model was then used to predict the outcome of the k^{th} training sample. The process was repeated k times, excluding a different training sample for validation each time.

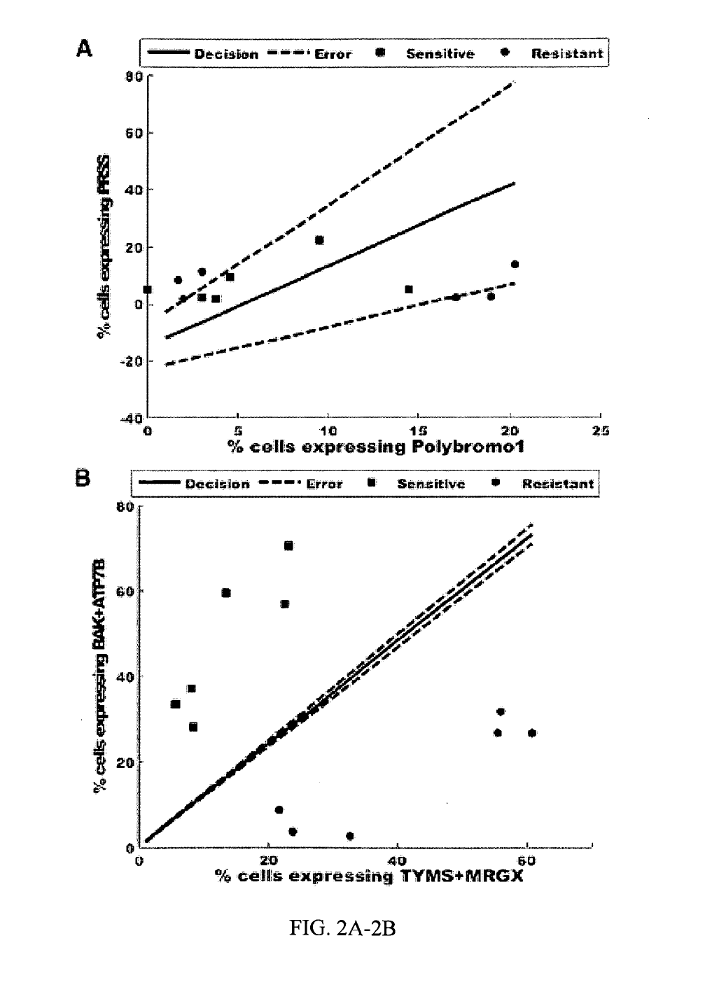

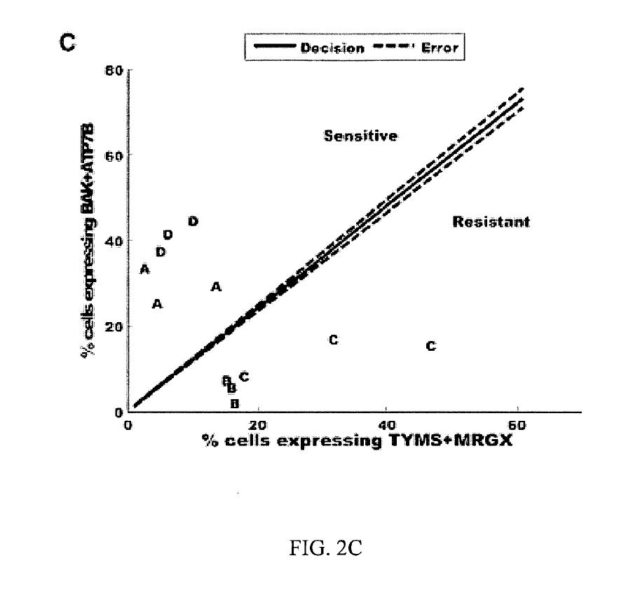

If a set of genes was not a good predictor of response to 5-FU, then the decision boundary was sensitive to each of the k training samples that were excluded. The result was a large variation between calculated decision boundaries, leading to poor sensitivity and specificity of the predictive model (FIG. 2A). Alternatively, FIG. 2B shows a set of genes whose expression levels yielded a model with high predictive accuracy and robustness. The variance between k decision boundaries calculated for each of the k subsets was small. A gene expression signature consisting of four genes, TYMS, MRGX, BAK and ATP7B correctly classified the training set of cell lines as either sensitive or resistant to 5-FU (FIG. 2B).

This model was then used to predict the response of independent test cell lines to 5-FU. Four additional colorectal adenocarcinoma cell lines, HCT15, SW620, RKO, and HCT116, were used to test the predictive model. Analysis of these test cell lines was blinded to eliminate bias in scoring of transcription sites. Cells were scored for number of transcription sites for MRGX, TYMS, BAK, and ATP7B. This model, consisting of these four genes, correctly predicted the response of all four test cell lines to 5-FU (FIG. 2C): SW620 (P=0.023) was classified as 5-FU-resistant while RKO (P=0.051) and HCT116 (P=0.0005) were classified as 5-FU-sensitive. The fourth cell line, HCT15 was classified as 5-FU-resistant with somewhat lower significance (P=0.099).

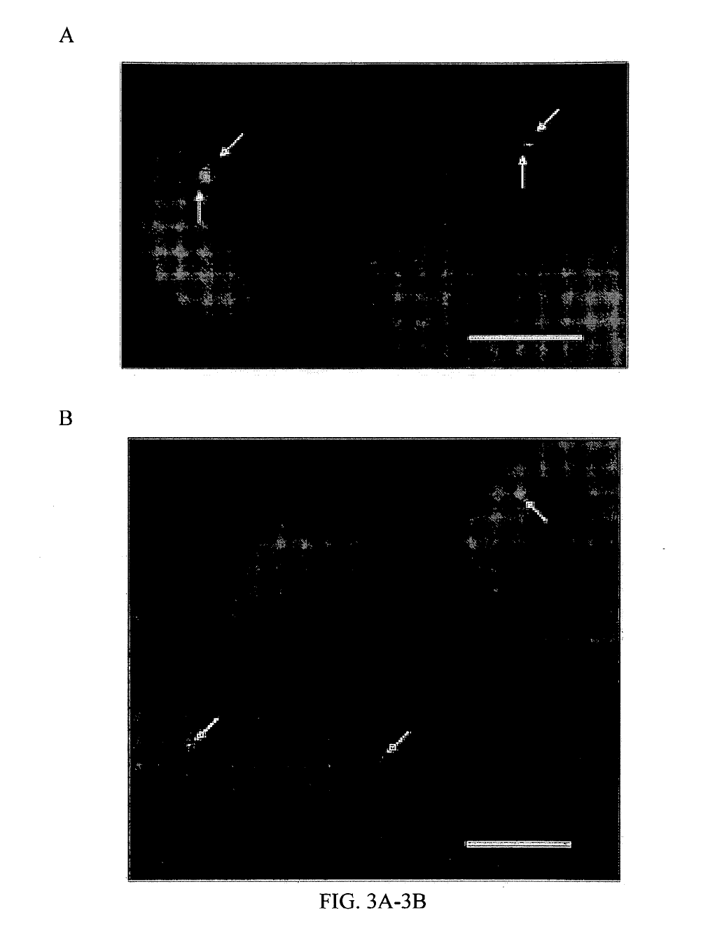

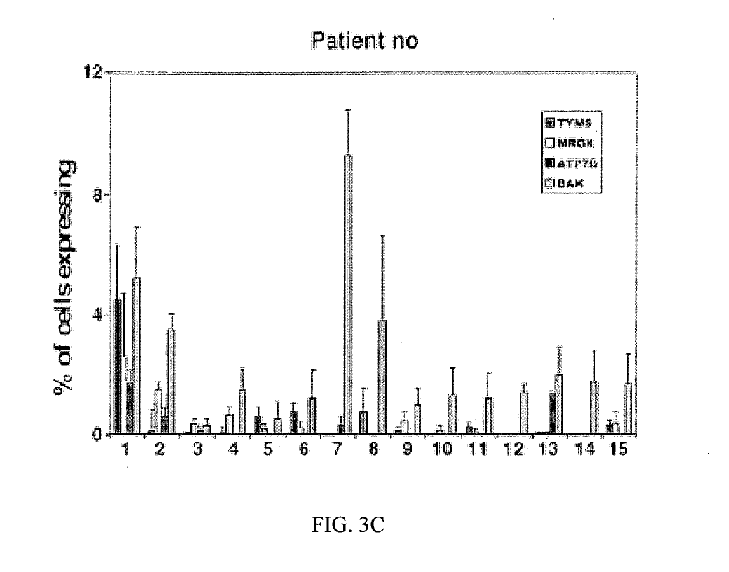

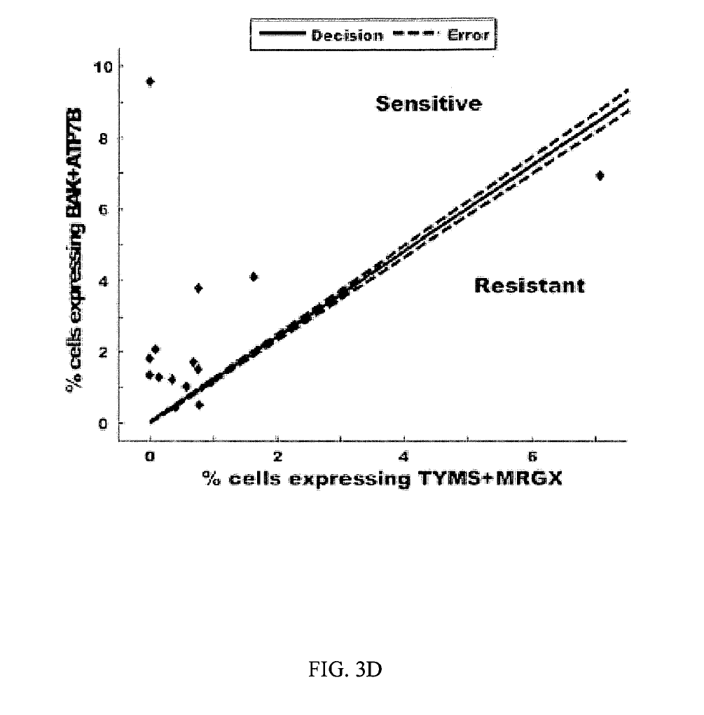

To investigate the potential of using transcription site profiling in tumors, active transcription sites in tissue samples from 15 anonymous colon cancer patients were examined on a TMA hybridized with probes for either TYMS and MRGX (FIG. 3A) or BAK and ATP7B (FIG. 3B). Although colon tumor tissues were all from patients with grade 2 colon adenocarcinomas, single-cell transcription site profiles of individual tumors revealed a large variability in the expression of marker genes (FIG. 3C). A majority of these tumor samples had high expression of the proapoptotic gene BAK, suggesting that these early grade tumors would be sensitive to apoptosis induced by chemotherapeutic drugs such as 5-FU. The predictive model classified 11 of the 15 samples as relatively sensitive (FIG. 3D). Two of the 15 tumors were classified as more resistant, while the remaining two tumors showed mixed characteristics.

To provide proof of principle that these transcription site profiles are associated with outcomes to therapy, colon tumor samples from a small number of patients with known outcomes were tested (Table 1).

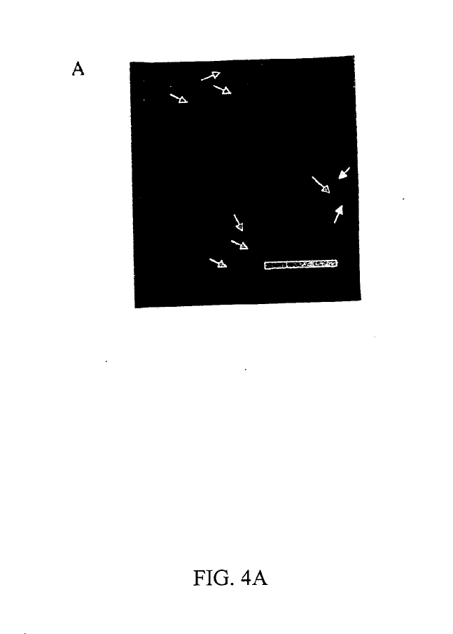

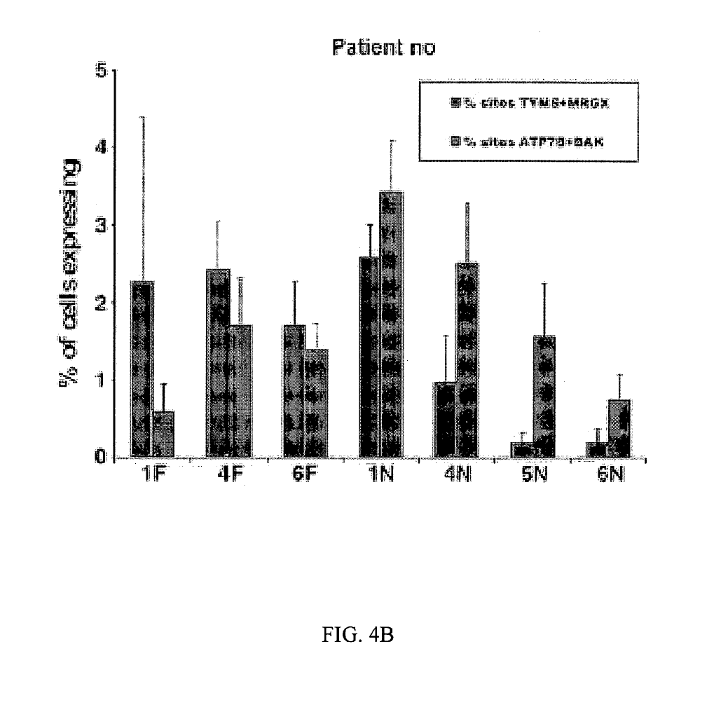

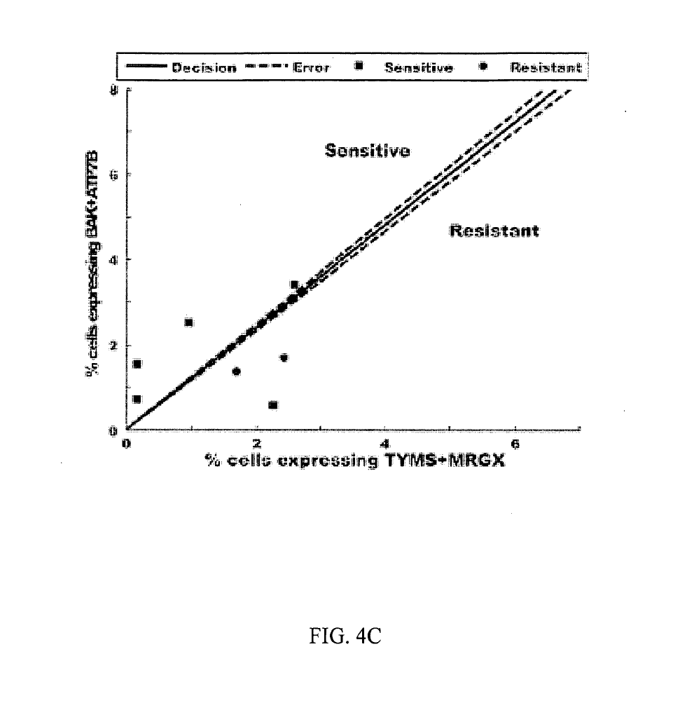

Tissue samples were obtained from surgically resected tumors of patients undergoing treatment for colon cancer. Three patients, designated 1F, 4F and 6F, received 5-FU-based chemotherapy before and after surgery, while four patients, designated 1N, 4N, 5N and 6N received 5-FU-based therapy only after surgery. Tissues were hybridized with probes for TYMS, MRGX, BAK and ATP7B (FIG. 4A). Analysis was blinded to eliminate bias in the scoring of transcription sites. Tumors from patients 1F, 4F and 6F had relatively higher expression of TYMS and MRGX and lower expression of ATP7B and BAK, classifying these patients as relatively less sensitive to 5-FU-based chemotherapy (FIG. 4B). Among these 3 patients, 1F had tumor recurrence following previous surgery and 5-FU-based chemotherapy, while patients 4F and 6F both later developed metastatic disease after 5-FU-based chemotherapy. In contrast, patients 1N, 4N, 5N and 6N had tumors with higher expression of ATP7B and BAK than TYMS and MRGX, classifying them as more sensitive to 5-FU-based chemotherapy (FIG. 4B). These four patients have not had a recurrence of their tumors or evidence of metastasis following surgery and 5-FU therapy, consistent with their classification as more sensitive to the drug treatment they received. On the basis of the predictive model, tumors from patients 1F, 4F and 6F were classified as relatively resistant and tumors from patients 1N, 4N, 5N and 6N as relatively sensitive (FIG. 4C).

### Discussion

Assay of transcription site activation differs from gene expression profiling in two key ways. First, expression analysis by Northern blots, qRT-PCR or microarrays measures steady-state transcript levels, while transcription site analysis provides data on whether the gene is on or off, essentially measuring the function of the gene as a rheostat that monitors and provides input into determining steady state levels. As such, transcription site analysis can provide insight into the presence or absence of signals and pathways that directly activate transcription. Second, by nature of the assay, transcription site analysis provides information on individual cells, rather than the mean level of expression of a gene in a population. Thus, this method is better suited for analysis of limited amounts of tissue and for dissection of heterogeneity that likely exists in tumors. Further, this FISH-based methodology can be combined with histopathology to provide a more accurate molecular picture of the cell biology of individual tumors, such as spatial distribution and orientation of tumor cells with particular phenotypes in the context of stromal-epithelial cell interactions (17), as well as the histopathological features of cells with particular transcription site profiles.

As discussed herein, it was shown that transcription site profiles of key genes for which steady state levels correlate with response to 5-FU in vitro (11) could be used to develop a novel approach that predicts response of tumor cells to chemotherapy. Using cell lines representing the extremes of sensitivity and resistance, a four-gene signature was derived that independently predicted 5-FU response in test cell lines. Further, by extending the analysis to human colon tumor tissue, proof of principle that the transcription site profile of cells varies among individual tumors was provided and may be used to predict patient response to 5-FU.

### REFERENCES

1. Longley D B, Harkin D P, Johnston P G. 5-fluorouracil: mechanisms of action and clinical strategies. Nat Rev Cancer 2003; 3(5):330-8.
2. Leichman C G, Lenz H J, Leichman L, et al. Quantitation of intratumoral thymidylate synthase expression predicts for disseminated colorectal cancer response and resistance to protracted-infusion fluorouracil and weekly leucovorin. J Clin Oncol 1997; 15(10):3223-9.
3. Salonga D, Danenberg K D, Johnson M, et al. Colorectal tumors responding to 5-fluorouracil have low gene expression levels of dihydropyrimidine dehydrogenase, thymidylate synthase, and thymidine phosphorylase. Clin Cancer Res 2000; 6(4):1322-7.
4. Metzger R, Danenberg K, Leichman C G, et al. High basal level gene expression of thymidine phosphorylase (platelet-derived endothelial cell growth factor) in colorectal tumors is associated with nonresponse to 5-fluorouracil. Clin Cancer Res 1998; 4(10):2371-6.
5. Elsaleh H, Iacopetta B. Microsatellite instability is a predictive marker for survival benefit from adjuvant chemotherapy in a population-based series of stage III colorectal carcinoma. Clin Colorectal Cancer 2001; 1(2):104-9.
6. Elsaleh H, Powell B, McCaul K, et al. P53 alteration and microsatellite instability have predictive value for survival benefit from chemotherapy in stage III colorectal carcinoma. Clin Cancer Res 2001; 7(5):1343-9.
7. Goh H S, Yao J, Smith D R. p53 point mutation and survival in colorectal cancer patients. Cancer Res 1995; 55(22):5217-21.
8. Benhattar J, Cerottini J P, Saraga E, Metthez G, Givel J C. p53 mutations as a possible predictor of response to chemotherapy in metastatic colorectal carcinomas. Int J Cancer 1996; 69(3):190-2.
9. Ahnen D J, Feigl P, Quan G, et al. Ki-ras mutation and p53 overexpression predict the clinical behavior of colorectal cancer: a Southwest Oncology Group study. Cancer Res 1998; 58(6):1149-58.
10. Arango D, Corner G A, Wadler S, Catalano P J, Augenlicht L H. c-myc/p53 interaction determines sensitivity of human colon carcinoma cells to 5-fluorouracil in vitro and in vivo. Cancer Res 2001; 61(12):4910-5.
11. Augenlicht L H, Wadler S, Corner G, et al. Low-level c-myc amplification in human colonic carcinoma cell lines and tumors: a frequent, p53-independent mutation associated with improved outcome in a randomized multi-institutional trial. Cancer Res 1997; 57(9):1769-75.
12. Mariadason J M, Arango D, Shi Q, et al. Gene expression profiling-based prediction of response of colon carcinoma cells to 5-fluorouracil and camptothecin. Cancer Res 2003; 63(24):8791-812.
13. Arango D, Wilson A J, Shi Q, et al. Molecular mechanisms of action and prediction of response to oxaliplatin in colorectal cancer cells. Br J Cancer 2004; 91(11):1931-46.
14. Levsky J M, Singer R H. Gene expression and the myth of the average cell. Trends Cell Biol 2003; 13(1):4-6.
15. Femino A M, Fay F S, Fogarty K, Singer R H. Visualization of single RNA transcripts in situ. Science 1998; 280(5363):585-90.
16. Levsky J M, Shenoy S M, Pezo R C, Singer R H. Single-cell gene expression profiling. Science 2002; 297(5582):836-40.
17. Capodieci P, Donovan M, Buchinsky H, et al. Gene expression profiling in single cells within tissue. Nat Methods 2005; 2(9):663-5.

1. A method for predicting resistance of a tumor to a chemotherapeutic agent comprising determining the level of expression of TYMS and/or MRGX, wherein a high level of expression of TYMS and/or MRGX indicates that the tumor is resistant to the chemotherapeutic agent.

2. The method of claim 1, wherein the tumor is a human colorectal tumor.

3. The method of claim 1, wherein the level of expression is determined by the number of active transcription sites for TYMS and/or MRGX.

4. The method of claim 3, wherein the number of active transcription sites is determined via fluorescence in situ hybridization.

5. The method of claim 3, wherein the active transcription sites are located in the interphase nucleus of the cell.

6. The method of claim 1, wherein the chemotherapeutic agent is 5-Fluorourcil (5-FU).

7. A method for predicting sensitivity of a tumor to a chemotherapeutic agent comprising determining the level of expression of ATP7B and/or BAK, wherein a high level of ATP7B and/or BAK indicates that the tumor is sensitive to the chemotherapeutic agent.

8. The method of claim 7, wherein the tumor is a human colorectal tumor.

9. The method of claim 7, wherein the level of expression is determined by the number of active transcription sites for TYMS and/or MRGX.

10. The method of claim 9, wherein the number of active transcription sites is determined via fluorescence in situ hybridization.

11. The method of claim 9, wherein the active transcription sites are located in the interphase nucleus of the cell.

12. The method of claim 7, wherein the chemotherapeutic agent is 5-Fluorourcil (5-FU).

13. A method for predicting resistance of a tumor to a chemotherapeutic agent comprising determining the level of expression in a cell of the tumor for TYMS and/or MRGX, and for ATP7B and/or BAK, wherein a higher level of expression for TYMS and/or MRGX compared to the level of expression for ATP7B and/or BAK indicates that the tumor is resistant to the chemotherapeutic agent.

14-18. (canceled)

19. A method for predicting sensitivity of a tumor to a chemotherapeutic agent comprising determining the level of expression in a cell of the tumor for TYMS and/or MRGX, and for ATP7B and/or BAK, wherein a lower level of expression for TYMS and/or MRGX compared to the level of expression for ATP7B and/or BAK indicates that the tumor is sensitive to the chemotherapeutic agent.

20-24. (canceled)

25. A method for determining the likelihood of tumor reoccurrence following treatment with a chemotherapeutic agent comprising determining the level of expression in a cell of the tumor for TYMS and/or MRGX, and for ATP7B and/or BAK, wherein a higher level of expression for TYMS and/or MRGX in the cell compared to the level of expression for ATP7B and/or BAK in the cell indicates that the tumor is likely to reoccur following treatment with the chemotherapeutic agent.

26-30. (canceled)

31. A method for determining the resistance or sensitivity of a tumor to a chemotherapeutic agent comprising:
(a) determining the level of expression in a cell of the tumor for a gene or genes known to correlate in response to the chemotherapeutic agent; and
(b) comparing the level of expression determined in step (a) with the level of expression in a cell of a control tumor having a known resistance or sensitivity to the chemotherapeutic agent,
wherein the resistance or sensitivity of the tumor to the chemotherapeutic agent is similar to that of the control tumor if the level of expression of the gene or genes in the cell of the tumor is similar to that of the cell of the control tumor.

32-37. (canceled)
